(19) United States
(12) Patent Application Publication
(10) Pub. No.: US 2017/0027486 A1
(43) Pub. Date: Feb. 2, 2017

(54) SYSTEM & METHOD FOR ESTIMATING SUBSTANCE CONCENTRATIONS IN BODILY FLUIDS

(71) Applicant: The City University, London (GB)

(72) Inventors: Panayiotis Kyriacou, London (GB); Meha Qassem, London (GB); Loukas Constantinou, London (GB); Iasonas Triantis, London (GB); Michelle Hickey, London (GB)

(73) Assignee: The City University, London (GB)

(21) Appl. No.: 15/302,784

(22) PCT Filed: Apr. 7, 2015
(86) PCT No.: PCT/EP2015/057527

§ 371 Date: Oct. 7, 2016

(30) Foreign Application Priority Data
Apr. 7, 2014 (GB) 1406257.4

(51) Int. Cl. A61B 5/145; A61B 5/053; A61B 5/16; A61B 5/00; A61B 5/1495; A61B 5/1455; A61B 5/1477

(52) U.S. Cl. CPC A61B 5/14546; A61B 5/1455; A61B 5/0537; A61B 5/1477; A61B 5/742; A61B 5/1495; A61B 5/165; A61B 2562/0238; A61B 2562/063

(57) ABSTRACT

One implementation described herein includes a system for providing at least an estimate of substance concentrations in a bodily fluid. The system comprises a first emitter for generating signals using a first sensing modality, a first detector configured to detect signals from said first emitter that have travelled through the bodily fluid and output a corresponding first detector signal; a second emitter for generating signals using a second sensing modality different to said first sensing modality, a second detector configured to detect signals from said second emitter that have travelled through the bodily fluid and output a corresponding second detector signal; and a processor configured to receive the detector signals from said first and second detectors, to process said signals and to output an indication of at least an estimate of the concentration of said substance in said bodily fluid.

### FIELD

This invention relates to systems and methods for estimating substance concentrations in bodily fluids. Illustrative embodiments of the present invention relate to systems and methods whereby substance concentrations in a bodily fluid, for example blood, can be estimated non-invasively and in vivo.

The teachings of the present invention will be described hereafter with particular emphasis on the estimation of substance concentrations in blood, particularly but not exclusively with emphasis on lithium concentration estimation. However, it will be immediately apparent to persons of ordinary skill in the art that the teachings of the present invention may be applied to the estimation of substance concentrations in a variety of different bodily fluids, and/or to a variety of different substances, and as such the following description should not be interpreted as being limited solely to the estimation of substance concentrations in blood or the estimation of lithium concentrations. It is also the case that whilst it is preferred for the estimation of substance concentrations to be accomplished in vivo, this is not essential and the system and method disclosed may equally well be employed for in vitro or ex vivo estimation of substance concentrations in bodily fluids. Lastly, it should be noted that whilst the arrangements disclosed herein may be capable of providing an accurate measurement of substance concentrations, it may be preferred for clinical reasons only to provide an indicator of the concentration (for example, low, normal or high) rather than an absolute value. The following detailed description and claims should be construed in the light of the foregoing.

### BACKGROUND

Bipolar disorder is a serious life-long disorder, often characterised by recurrent episodes of depression and mania. In its more severe forms, bipolar disorder is associated with significant impairment of personal and social functioning, and with high risk of death through suicide as well as poor physical health. About one to two percent of the population in the UK has been diagnosed with bipolar disorder, and it has been estimated that the annual societal cost of bipolar disorder in the UK is about £2 billion.

Lithium is the most widely used medication for treating bipolar disorder, and although it is highly effective at reducing the frequency and intensity of mood swings, it can be potentially dangerous. Lithium prescribed in the form of carbonate or citrate has a very narrow therapeutic range (concentrations ranging from 0.4 to 1.0 mmol/L) with the upper limit being uncomfortably close to toxic levels. The use of lithium salts can affect thyroid and kidney function, and toxic lithium levels can cause circulatory collapse, kidney failure, neurological abnormalities, seizures, coma and even death.

Whilst a person is in good health, lithium concentrations tend to be reasonably stable, however they can rapidly reach toxic levels during intercurrent illness such as febrile conditions and dehydration or the addition of some drugs.

In view of the foregoing, lithium requires regular on-going monitoring to maintain therapeutic levels and avoid toxicity. Current techniques for determining blood lithium levels involve relatively complex laboratory methods, such as flame photometry or ion-selective electrode analysis, which require withdrawal of blood samples and transport of samples to the laboratory.

The National Institute for Health and Clinical Excellence (NICE) guidelines recommend that lithium levels should be checked one week after starting and one week after every dose change until the levels are stable. This process of dosage adjustment can take months before stability is reached. Following stability, NICE recommends that lithium levels are checked every three months, along with regular monitoring of kidney and thyroid function. However, in a national-level audit of lithium monitoring practice in the UK, it was found that contemporary lithium monitoring falls short of the standards recommended by NICE.

This failure to ensure the safe use of lithium and/or to ensure adequate monitoring of established treatment may place subjects at risk of avoidable drug related morbidity. The difficulties in ensuring lithium monitoring in its current form may include the practicalities of having to attend a clinic for blood sampling and dislike/fear of having blood taken. Consequently bipolar subject non-adherence with lithium and lithium toxicity are serious issues that need to be addressed. Currently, there is no commercially available, portable non-invasive system that would enable subjects suffering from Bipolar Disorder to personally monitor their own lithium levels and thereby quickly spot when their lithium concentration appears to be drifting outside therapeutic ranges.

An associated problem is that as lithium generally has a very low concentration in blood (concentrations ranging from 0.4 to 1.0 mmol/L), such a system would need to relatively sensitive to provide adequate measurement accuracy. This is particularly the case when one considers the fact that there will likely be interfering analytes and environments that differ from subject to subject (e.g. different types of skin, blood content affected by other conditions etc).

The present invention has been devised with the foregoing in mind.

### SUMMARY

In accordance with a presently preferred embodiment of the present invention, there is provided a system for estimating substance concentrations in bodily fluids, the system comprising: a first component for generating signals using a first sensing modality, and a second component for generating signals using a second different sensing modality, wherein the signals generated by said first and second components are each representative of the concentration of a substance in a bodily fluid, and the system further comprises a processor for estimating concentration of said substance in said bodily fluid from the signals generated by said first and second components.

In one implementation, the first component is configured to use an optical sensing modality, and the second component is configured to use an electrical sensing modality. The first component may comprise one or more optical sources and an optical detector. The second component may comprise a plurality of electrodes.

Another aspect of the invention relates to a method for estimating substance concentrations in bodily fluids, the method comprising: operating a first component to generate signals using a first sensing modality, operating a second component to generating signals using a second different sensing modality, wherein the signals generated by said first and second components are each representative of the concentration of a substance in a bodily fluid, and operating a processor to estimate the concentration of said substance in said bodily fluid from the signals generated by said first and second components.

A further aspect of the invention relates to a system for providing at least an estimate of substance concentrations in a bodily fluid, the system comprising: a first emitter for generating signals using a first sensing modality; a detector configured to detect signals from said first emitter that have travelled through the bodily fluid and output a corresponding first detector signal; a second emitter for generating signals using a second sensing modality different to said first sensing modality; a second detector configured to detect signals from said second emitter that have travelled through the bodily fluid and output a corresponding second detector signal; and a processor configured to receive the detector signals from said first and second detectors, to process said signals and to output an indication of at least an estimate of the concentration of said substance in said bodily fluid.

The system may comprise a control module, and a probe coupled to the control module for communication therewith. The control module may comprise a display controllable by the processor to display an indication of at least an estimate of the concentration of said substance in said bodily fluid. The indication may be coloured and the colour of said indication may vary with estimated substance concentrations. The indication may be text-based, and the text may vary with variations in estimated substance concentrations.

The control module and probe may be wirelessly coupled to one another. The control module may be hand-holdable and portable.

The probe may comprise the first and second emitters and the first and second detectors. The probe may be configured to be adhered or otherwise affixed to a subject.

In one embodiment the first sensing modality comprises an optical sensing modality. The first emitter may be operable to emit light of one or more wavelengths.

The first emitter may be operable to emit light at a plurality of wavelengths. The first emitter may comprise at least a first and a second LED. The first detector signal may be representative of the intensity of light received from said first emitter.

In one embodiment, the second sensing modality comprises an electrical sensing modality. The second emitter may comprise a plurality of electrical contacts. The system may be configured to apply a current to a bodily fluid at one or a plurality of frequencies. The second detector signal may be representative of the impedance of the bodily fluid.

The processor may, in one embodiment, be provided with a calibration model configured to output an estimate of substance concentration when the model is provided with said first and second detector signals. The substance may comprise lithium.

Another aspect of the present invention relates to a method for providing at least an estimate of substance concentrations in a bodily fluid, the method comprising: operating a first emitter to generate signals using a first sensing modality, operating a detector to detect signals from said first emitter that have travelled through the bodily fluid, and outputting a corresponding first detector signal; operating a second emitter to generate signals using a second sensing modality different to said first sensing modality, operating a second detector to detect signals from said second emitter that have travelled through the bodily fluid, and outputting a corresponding second detector signal; and providing a processor that is configured to receive the detector signals from said first and second detectors, and operating said processor to process said signals and to output an indication of at least an estimate of the concentration of said substance in said bodily fluid.

### BRIEF DESCRIPTION OF THE DRAWINGS

Various aspects of the teachings of the present invention, and arrangements embodying those teachings, will hereafter be described by way of illustrative example with reference to the accompanying drawings, in which:

FIG. 1 is a schematic representation of a system for monitoring substance concentrations;

FIG. 2 is a schematic plan view of a probe for use with the system of FIG. 1;

FIG. 3 is a schematic representation of an adhesive attachment configured for attaching the probe of FIG. 2 to a subject to be monitored;

FIG. 4 is a schematic representation of the components of the system;

FIG. 5 is a representative circuit diagram for components of a control module for the system of FIG. 1; and

FIG. 6 is a schematic representation of an in-vitro device for monitoring substance concentrations.

### DETAILED DESCRIPTION

In very general terms, one envisaged arrangement provides a system for monitoring substance concentrations in a bodily fluid, the system comprising a control module and a probe that is coupled to the control module. To provide an adequate level of sensitivity the probe utilises at least two different sensing modalities. The system can be used in vitro (for example on blood spot or saliva samples), ex vivo, or in vivo (for example transcutaneously or indwelling), for example to enable continuous monitoring of substance concentration. The bodily fluid could comprise blood, saliva, sweat or urine, and the substance whose concentration is being estimated could be lithium.

A probe configured for in vitro use sits over a small platform where the blood or saliva sample is placed. The interface between the fluid sample and the sensors components comprises a disposable attachment. A similar arrangement is provided for ex vivo sensing of analyte concentration.

A probe that is configured for in vivo use can be designed to fix onto an appendage of a subject's body, such as the finger, ear or lip, or be affixed (for example adhered) to some other suitable part of the subject. In one envisaged arrangement, the probe can be held in place on the appendage by gentle pressure exerted by a spring-clip, but other equally appropriate fixing mechanisms, such as medical adhesive tape, could instead be used.

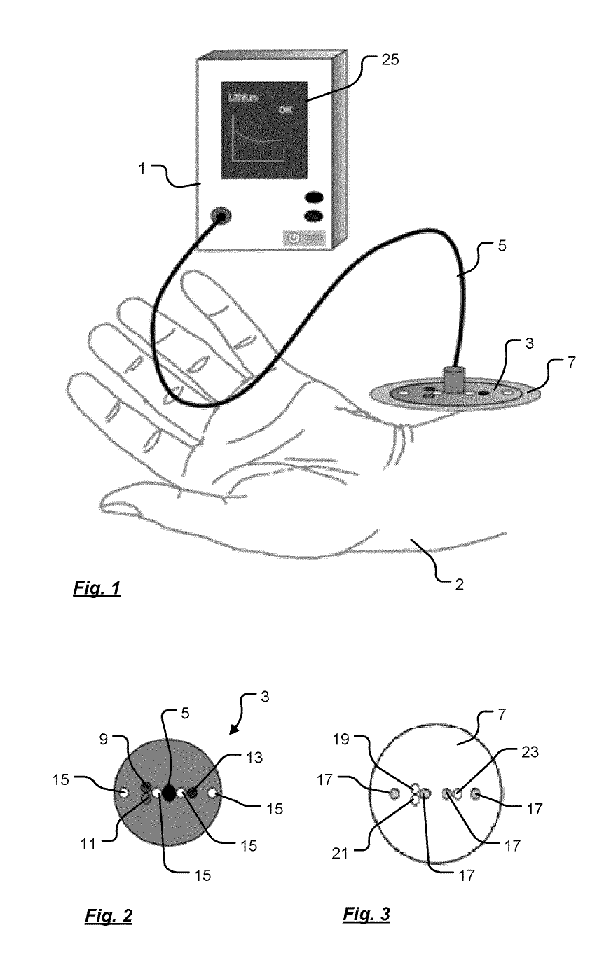

FIG. 1 is a schematic representation of a system for in vivo estimation of substance concentration. As shown, the system comprises a control module 1 and a probe 3. The probe 3 is coupled, in this case by means of a wire 5 to the control module 1 (note that the probe 3 could be wirelessly coupled to the control module, in which case the probe would be provided with its own power supply), and is attachable to a subject 2 by means of an adhesive attachment 7 that can be adhered to the subject and to the underside of the probe 3. In one envisaged implementation the probe is configured to be flexible so that it can conform more readily to the area of the subject to which it is to be attached.

FIG. 2 is a top plan view of the probe 3. As shown, the probe 3 is coupled to the control module 1 by means of the cable 5. As aforementioned, the probe includes appropriate emitters and detectors for implementing two different sensing modalities—in this particular example optical and electrical. The probe 3 of this particular embodiment comprises first and second light sources 9, 11 (for example, light emitting diodes or lasers) that—in the preferred implementation—are operable to emit light of different wavelengths. The probe further comprises a photodetector 13 for detecting light emitted by the sources that has travelled transcutaneously (in this example) through the subject.

In addition to the aforementioned optical components, the probe 3 also comprises emitters and detectors for a second different sensing modality. In this particular example the probe 3 comprises an array of electrodes 15, in this particular example four electrodes 15 that are evenly spaced along a diameter of the probe.

FIG. 3 is a schematic plan view of an adhesive attachment 7 for adhering the probe to a subject. The attachment 7 includes a layer of adhesive on its upper side (so that the attachment can be adhered to the underside of the probe) and on its lower side (so that the attachment can be adhered to a subject. The attachment 7 further comprises an array of (in this instance four) electrically conducting contacts 17 that align with and electrically contact the electrodes 15 on the probe 3 when the attachment 7 is adhered thereto, first and second optical windows 19, 21 that align with the first and second light sources 9, 11 and a third optical window 23 that aligns with the photodetector 13.

Whilst FIGS. 1 to 3 of the drawings depict a probe for in vivo use, it should be noted that the technical properties and configuration of the in vivo, ex vivo and in vitro probes are largely identical. The probe consists of emitters and detectors for at least two different sensing modalities, for example optical components and electrical components (for optical and electrical modalities, respectively). The optical components comprise at least one and preferably multiple light sources (for example, light emitting diodes or lasers) that emit light of appropriate wavelengths in the UV-VIS-NIRS range for the detection of lithium, or other wavelengths for alternative analytes of interest. The light sources are arranged to illuminate the subject (for example an appendage of the subject) or the in vitro fluid sample, and the optical component further comprises a light detector (for example a photodiode) that is sensitive to the particular wavelengths of light emitted by the source. In one implementation the light detector is provided on the same side of the appendage/sample as the light sources—in what is known as a reflection mode. However, in another implementation the light detector may be placed opposite the light sources to interrogate the samples in what is known as a transmission mode.

In one particularly preferred implementation one or two optical emitters will be provided for emitting light at UVNisible/IR wavelengths specific for lithium, or a marker of lithium (or other analyte of interest) absorption. It is also envisaged to provide an emitter that emits light of another wavelength, such as visible red light of approximately 660 nm, so that an indication of the blood volume being sampled can be obtained (such an indication then being used to estimate the lithium or other analyte concentration).

In summary, the light sources illuminate the appendage of the subject or in vitro sample with light wavelengths that enable the appropriate identification of lithium (or other analyte of interest) and the detector generates a signal (in particular a photocurrent) that varies in dependence upon the amount of incident light that is absorbed as the light traverses the appendage/sample.

As will later be described in detail, in one implementation the light sources and detector of the probe 3 are coupled to the control module 1 so that the control module can drive the sources, time multiplex the sources so that they are not on at the same time, and determine the intensity of light detected by the detector.

The electrical component of the probe comprises of more than two, and preferably but not exclusively four metallic electrodes. In a preferred arrangement, the electrodes are located in the vicinity of the optical sources and detector so that the optical and electrical components of the system consider approximately the same region of subject tissue. It will be appreciated, however, that whilst this arrangement is preferred the electrical and optical components of the probe could merely be in close proximity to one another, for example adjacent to one another, or able to operate independently and at distant locations to one another, if these are deemed to offer complementary information about a medical condition of interest.

In an envisaged implementation two electrodes supply alternating current at an appropriate current injection magnitude within the biocompatible range (100 microamps from 0.1 Hz to 1 kHz; then 100*f microamps from 1 kHz to 100 kHz (where f is the frequency in kHz); then 10 mA above 100 kHz) into the tissue or sample. In an envisaged implementation the current injection frequencies are programmable with the option of combining multiple frequency components on a single waveform, as each may provide information for different types of tissue and/or analytes. In a preferred arrangement, at least one of the frequencies will be appropriate for lithium detection and analysis, by both instantaneous and dynamic measurements. Multi-frequency current injection to the sample will also provide accurate, rapid measurement. Two electrodes, preferably but not exclusively different to the current injection ones, will sense the generated voltage in the tissue/sample.

As will later be described in detail, the electrodes are coupled to the control module so that the control module can—in a preferred but not exclusive arrangement—provide the appropriate drive current to the injection electrodes, provide single or multi-frequency synchronous detection, and determine phase and amplitude measurements from the sensing electrodes (note that phase and amplitude measurements can be performed preferably but not exclusively with the use of a synchronous detection technique).

The spacing between the electrodes, as well as their contact area and material allow for accurate interrogation of the sample and control of the depth of measurement. As mentioned above, in one implementation the probe 3 is wired to the control module 1. The wire 5 comprises a multi-strand cable that electrically connects the light sources 9, 11, the photodetector 13, the two injection electrodes and the two sensing electrodes 15 to the control module 1. Preferably, the cable is shielded to reduce electromagnetic interference.

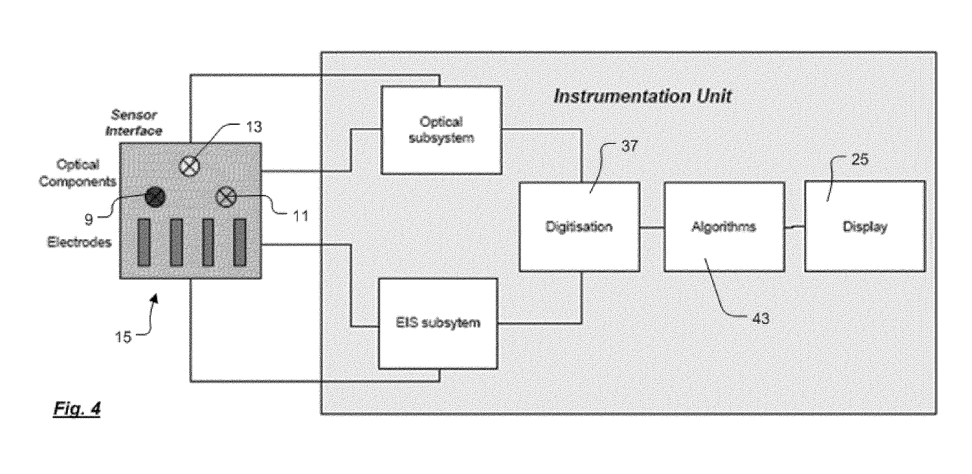

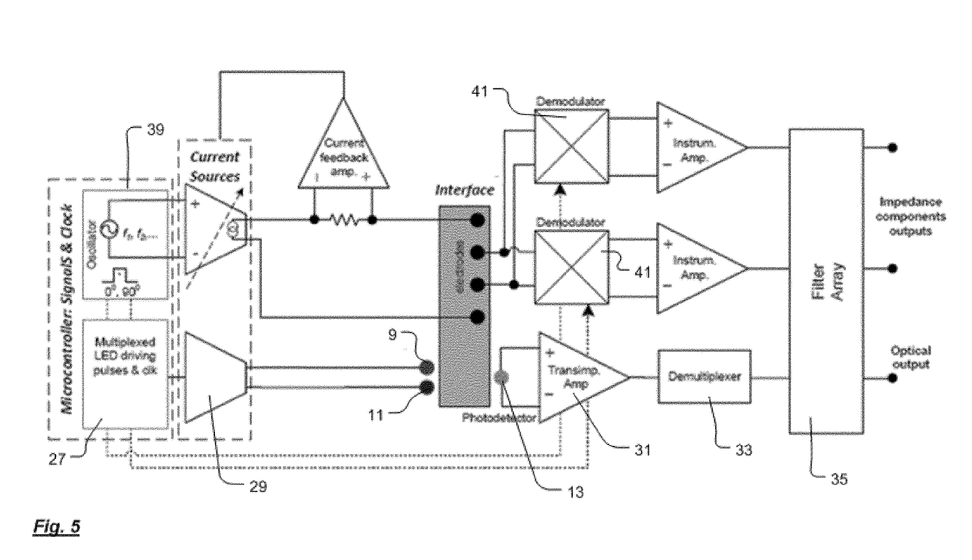

FIGS. 4 and 5 are schematic representations of the control module 1. It is envisaged that the control module will be embodied as a relatively small and readily portable unit that can be coupled to the probe. For example, the control module 1 may be configured as a hand-holdable, battery powered portable device with an integrated display 25 (FIG. 1). In another envisaged implementation, the control module may be configured for desktop use, and in this configuration will likely be configured to draw power from the mains electrical supply.

The control module contains a power supply (for example a low voltage battery or suitable alternative, not shown) that powers the control module as a whole, and the components in the probe. The control module consists of optical and electrical impedance subsystems which work together in powering both the optical and electrical probe components and ensuring adequate multiplexing for sample interrogation.

The optical spectroscopy subsystem contains a multiplexer circuit 27 to generate the clock signals to control switching on and off the light sources. These signals control a controllable current source 29 that drives the light sources 9, 11 with the required current, and hence, allows for light intensity to be controlled. The light detector 13 outputs a signal to a circuit 31 that converts the generated current from the detector into a voltage. A demultiplexer 33 splits the signal into corresponding signals for each light wavelength. These signals are then processed by filters 35 and amplifiers and passed to an analogue to digital converter 37 (FIG. 4).

In one envisaged implementation, the electrical impedance subsystem includes on-board, controllable oscillators 39 forming the back-end of the signal injection stage.

The oscillators 39 generate the desired signals that will drive current sources, as well as generate the zero and ninety degree phase shifted demodulation signals that will “lock” the voltage measuring channels to the pre-allocated frequencies. These signals can consist of a single frequency or number of frequencies and will be used to control an ac current source for the electrical impedance electrodes, with closed-loop amplitude stabilisation. The current source will generate the required current signals, which will be injected through electrodes 15. Closed-loop amplitude stabilisation will be used to monitor the injected current signals and provide a correction signal to a separate input on the current source so as to adjust the output current signal. This will allow for accurate control of both the current injection magnitude and frequency. The current sources are wideband ac current sources with high output impedance to allow for measurement accuracy over a range of different samples and over a range of signal frequencies.

The signal from the sensing electrodes is then demodulated by demodulators 41 and generated demodulation signals, followed by an amplification stage to extract the voltage signal pertaining to sample information, in this case to extract the real and imaginary components of the signal. The system consists of two such channels per injected frequency. Extraction of impedance characteristics can be performed but not limited to extraction of the real and imaginary components of the signal as described in this case and is not limited to the particular arrangement set out herein. These signals are then processed by filters 35, amplifiers and dc-offset cancellation techniques and then passed to an analogue to digital converter 37.

The analogue to digital converter 37 outputs to a microprocessor 43 digital signals representative of the absorption of light by the sample and digital signals representative of the electrical impedance of the sample. The processor calculates from these signals the concentration of the analyte of interest (for example, lithium) and controls the display 25 to provide an indication of the calculated concentration, preferably along with a graphical representation of trend data indicating how that component concentration has varied over time. The digital data can be transmitted, displayed and post-processed for examining cross-sensor analyte-indicative correlations or mutual exclusions, as well as dynamic changes over time. A wired or alternatively a wireless transmitter offers the possibility for uploading data to memory storage or to a computing device with the possibility of directly updating the medical records of the subject and providing remote monitoring access to the clinician responsible.

As mentioned above, the analogue to digital converter 37 outputs to a microprocessor 43 digital signals representative of the absorption of light by the sample and digital signals representative of the electrical impedance of the sample. In a preferred implementation of the teachings of the invention, the processor 43 is provided with a multi-variate calibration model that is used to provide calculations of concentrations. The model is a type of regression model, such as a partial-least-squares (PLS) model, that enables current values to be estimated based on past data. The multi-variant model programmed on the micro-processor is based on in-vitro laboratory measurements on blood solutions with various concentrations of the anylyte of interest, for example lithium. The digital signals representing the light absorption and electrical impedance of the sample are fed into the model (which is held in the memory of the microprocessor), processed and the processor outputs a relative or absolute concentration of lithium, either in numerical form or as an indicator, such as a traffic light or text-based indicator.

In one envisaged implementation, the control module may be provided with a predetermined “high” threshold that corresponds to a concentration of the analyte of interest (for example, lithium) that is considered to be too high for the subject, and a predetermined “low” threshold corresponding to a concentration of the analyte of interest (for example, lithium) that is considered to be too low for the subject. These thresholds may be generic, or more preferably tailored to the particular needs of a given subject. In one envisaged implementation, the thresholds could be programmed into the control module by the medical professional charged with caring for the subject.

In such an implementation, the control module (in addition or as an alternative to the display of an indication of the calculated concentration and/or trend data) may be configured to display substance concentrations graphically, for example via a traffic light system where a red light indicates that the concentration is at or above the high threshold, a green light indicates that the concentration is between the high and low thresholds, and an amber light incidates that the concentration is at or below the low threshold. In another envisaged arrangement, the control module may additionally or alternatively be configured to provide audible warnings (optionally, different audible below the “low” threshold. A variety of alternative or additional ways of alerting the subject will be apparent to persons of ordinary skill in the art, for example a simple text alert (e.g. “lithium too low”, “lithium normal” or “lithium too high”) may instead or additionally be provided.

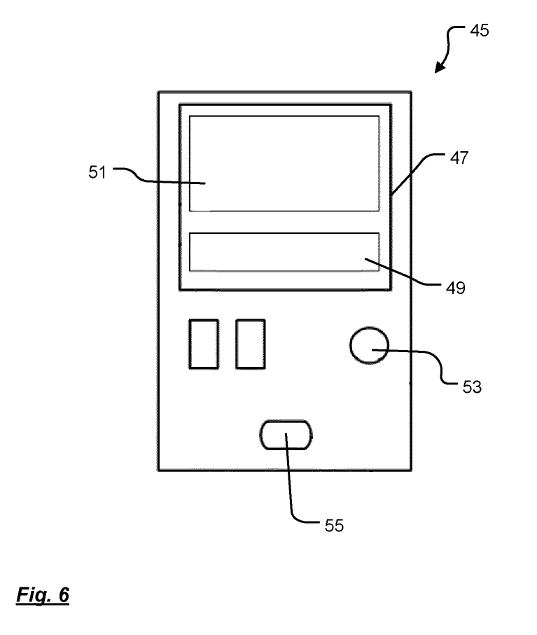

As previously mentioned, the present invention can be embodied as an in vivo device or as an in vitro device. FIG. 6 is a schematic representation of an illustrative in vitro device 45. The device 45 comprises a housing 47 in which a screen 47 is mounted. The screen is sub-divided into a first region 49 in which the concentration of the analyte of interest is displayed, and a second region 51 in which a green, red or amber icon is displayed to indicate, respectively, a normal, high or low analyte concentration. The device includes an on-off switch 53, and a sample entry port 55 in which a sample to be tested is deposited. The sample entry port may be a receptacle that is wiped-clean after each use, or may be configured to hold a disposable receptacle that is discarded once a given sample has been tested. The device is preferably hand-holdable, and battery powered. In contrast to the in vivo device depicted in FIG. 1, no probe is required.

It will be appreciated that whilst various aspects and embodiments of the present invention have heretofore been described, the scope of the present invention is not limited to the particular arrangements set out herein and instead extends to encompass all arrangements, and modifications and alterations thereto, which fall within the spirit and scope of the invention.

It should also be noted that whilst particular combinations of features are described herein, the scope of the present invention is not limited to the particular combinations hereinbefore described, but instead extends to encompass any combination of features herein disclosed. Finally, it should be noted that any element in a claim that does not explicitly state “means for” performing a specified function, or “steps for” performing a specific function, is not to be interpreted as a “means” or “step” clause as specified in 35 U.S.C. Sec. 112, par. 6.

1. A system for providing at least an estimate of substance concentrations in a bodily fluid, the system comprising:
a first emitter for generating signals using a first sensing modality,
a first detector configured to detect signals from said first emitter that have travelled through the bodily fluid and output a corresponding first detector signal;
a second emitter for generating signals using a second sensing modality different to said first sensing modality,
a second detector configured to detect signals from said second emitter that have travelled through the bodily fluid and output a corresponding second detector signal; and
a processor configured to receive the detector signals from said first and second detectors, to process said signals and to output an indication of at least an estimate of the concentration of said substance in said bodily fluid.

2. The system according to claim 1, comprising a control module, and a probe coupled to the control module for communication therewith.

3. The system according to claim 2, wherein the control module comprises a display controllable by the processor to display an indication of at least an estimate of the concentration of said substance in said bodily fluid.

4. The system according to claim 3, wherein said indication is coloured and the colour of said indication varies with estimated substance concentrations.

5. The system according to claim 3, wherein said indication is text-based, the text varying with variations in estimated substance concentrations.

6. The system according to claim 2, wherein the control module and probe are wirelessly coupled to one another.

7. The system according to claim 2, wherein the control module is hand-holdable and portable.

8. The system according to claim 2, wherein the probe comprises the first and second emitters and the first and second detectors.

9. The system according to claim 8, wherein said probe is configured to be adhered or otherwise affixed to a subject.

10. The system according to claim 1, wherein said first sensing modality comprises an optical sensing modality.

11. The system according to claim 10, wherein said first emitter is operable to emit light of one or more wavelengths.

12. The system according to claim 11, wherein said first emitter is operable to emit light at a plurality of wavelengths.

13. The system according to claim 12, wherein said first emitter comprises at least a first and a second LED.

14. The system according to claim 10, wherein said first detector signal is representative of the intensity of light received from said first emitter.

15. The system according to claim 1 wherein said second sensing modality comprises an electrical sensing modality.

16. The system according to claim 15, wherein said second emitter comprises a plurality of electrical contacts.

17. The system according to claim 15, wherein said system is configured to apply a current to a bodily fluid at one or a plurality of frequencies.

18. The system according to claim 15, wherein said second detector signal is representative of the impedance of the bodily fluid.

19. The system according to claim 1, wherein said processor is provided with a calibration model configured to output an estimate of substance concentration when the model is provided with said first and second detector signals.

20. The system according to claim 1, wherein said substance comprises lithium.

21. A method for providing at least an estimate of substance concentrations in a bodily fluid, the method comprising:
operating a first emitter to generate signals using a first sensing modality, operating a first detector to detect signals from said first emitter that have travelled through the bodily fluid, and outputting a corresponding first detector signal; operating a second emitter to generate signals using a second sensing modality different to said first sensing modality,
operating a second detector to detect signals from said second emitter that have travelled through the bodily fluid, and outputting a corresponding second detector signal; and
providing a processor that is configured to receive the detector signals from said first and second detectors, and operating said processor to process said signals and to output an indication of at least an estimate of the concentration of said substance in said bodily fluid.
